(19) United States
(12) Patent Application Publication
(10) Pub. No.: US 2018/0009662 A1
(43) Pub. Date: Jan. 11, 2018

(54) INHERENTLY SAFE OXYGEN/HYDROCARBON GAS MIXER

(71) Applicant: NOVA Chemicals (International) S.A., Fribourg (CH)

(72) Inventors: Vasily Simanzhenkov, Calgary (CA); Shahin Goodarznia, Calgary (CA); Kamal Elias Serhal, Calgary (CA); Bolaji Olayiwola, Calgary (CA); Eric Clavelle, Calgary (CA)

(73) Assignee: NOVA Chemicals (International) S.A., Fribourg (CH)

(21) Appl. No.: 15/628,671

(22) Filed: Jun. 21, 2017

(30) Foreign Application Priority Data
Jul. 7, 2016 (CA) 2935564

(51) Int. Cl. C01B 3/36; B01F 5/06; C07C 1/02; C01B 3/02; B01J 8/00

(52) U.S. Cl. CPC C01B 3/36; C07C 1/02; C01B 3/02; B01F 5/06; B01J 8/00; C01B 2203/1276; C01B 2203/02

(57) ABSTRACT

Provided herein is a gas mixer for the safe mixing of a hydrocarbon containing gas with a gaseous oxidant. The gas mixer and method for mixing described includes a closed mixing vessel where bubbles of gas injected at the bottom of the vessel are mixed during their rise to the top of the vessel, forming a homogeneous mixture that can safely be removed. This simple design and method allows for safe mixing of gases and is applicable to catalytic oxidative processes such as oxidative dehydrogenation of paraffins where there is a risk of thermal runaway of reactions.

This present disclosure relates to a system for the safe homogenous mixing of gases. More specifically a system for the safe mixing of a hydrocarbon gas, such as ethane, with a gaseous oxidant in a manner that risk of an explosive event is minimized is disclosed. Additionally, some embodiments are directly applicable to use in the catalytic oxidative dehydrogenation of ethane into ethylene.

Mixing of a hydrocarbon containing gas with a gaseous oxidant is associated with potentially catastrophic consequences. When the ratio of hydrocarbon to oxygen within a mixture is within the flammability envelope, ignition can result in a runaway reaction that if under pressure can lead to an explosive event. For processes where mixing of hydrocarbons with oxygen is required, consideration of how to minimize the likelihood of ignition is always a primary consideration. While processes for catalytic oxidation of hydrocarbons, for example oxidative dehydrogenation (ODH) of paraffins into olefins, are typically performed with mixtures of hydrocarbons and oxygen that are outside the flammability envelope there is still potential for ignition when the mixture is heterogeneous and includes hotspots where the ratio of hydrocarbon to oxygen is within explosive limits. This is particularly true when the components are in the initial phase of mixing before homogeneity is achieved.

It is commonly known that for ignition to occur there must be both a mixture of hydrocarbon and oxygen within the flammability envelope and an ignition event. An ignition event may take the form of entrained particles present within either the hydrocarbon or gaseous oxidant striking a metallic surface within the mixing apparatus and creating a spark. If the spark occurs in a region near where streams of the hydrocarbon gas and gaseous oxidant meet and have not reached homogeneity outside flammability limits, ignition may result. With that in mind, it is not surprising that prior art options for minimizing the risk of an explosive event, when mixing hydrocarbons and oxygen, are mainly focused on either decreasing the chances of an ignition causing spark, or by maximizing the rate of mixing to shorten the window when ignition may occur due to the existence of heterogeneous pockets of unfavorable hydrocarbon/oxygen compositions.

U.S. Pat. No. 8,404,189, issued Mar. 26, 2013 to Andresen et al., assigned to Dow Technology Investments LLC, claims a gas mixer that encompasses a vessel containing a flow of a first gas to be mixed, and an internal pipe within the vessel that contains the second gas to be mixed. The second gas is introduced into the flow of the first gas within the vessel via a mixer tip on the internal pipe. The mixer tip includes internal passageways that permit passage of the second gas out of the pipe and into the vessel in a radial plane at an acute angle relative to the longitudinal axis of the vessel and the internal pipe. The exit point on the mixer tip is aligned with an upstream deflector, which acts to deflect and minimize the risk of entrained particles within the first gas from striking the mixer tip, which is within the region where initial mixing of the gases occurs. Furthermore, the mixer tip may be comprised of a spark resistant material. Unfortunately there is still a possibility that an ignition may occur and the patent does not teach quenching of such an eventuality.

U.S. Pat. No. 8,500,894, issued Aug. 6, 2013 to Andresen et al., assigned to Dow Technology Investments LLC, teaches the use of a wet scrubber in the oxygen supply line to remove particulate matter before mixing with a hydrocarbon stream. By removing particulate matter there is a reduced risk of igniting the hydrocarbon gas stream during initial mixing with the oxygen stream. Wet scrubbers are known in the art and comprise passing the oxygen through a water medium so that entrained solid particles transfer to the aqueous phase where they can be removed easily. While minimizing the risk of ignition by reducing the number of entrained particles the patent does not teach the quenching of any ignition that may still occur.

U.S. Pat. No. 4,256,604 issued Mar. 17, 1981 to Aida et al., assigned to Nippon Shokubai Kagaku Kogyo Co., Ltd., claims a method for mixing gases that includes passing hydrocarbon gas through an aqueous medium before introduction of oxygen and subsequent mixing in a gas mixing zone, with the resulting mixture passing through a shielding zone before recycled back to an oxidation reactor. The aqueous medium and the shielding zone comprise trays present within the reactor that hold and ultimately guide water, or ethylene glycol in a counter current direction relative to the inputted hydrocarbon gas. The design is aimed at preventing back flow or forward movement of ignition events should they occur. However, the gas mixing zone may still suffer damage should ignition energy be generated allowing combustion to take place.

U.S. Pat. No. 6,657,079, issued Dec. 2, 2003, to Mitsumoto et al., assigned to Nippon Shokubai co., Ltd., claims a method for mixing a plurality of gases by using at least one gas inlet nozzle that is disposed to permit a helical flow pattern within the mixing vessel. The helical flow pattern results in the substantial elimination of internal items within the vessel allowing homogeneous mixing using exceptionally simple construction. Despite the advantage of simplicity there is still a risk of ignition and there is no mechanism for quenching of flammable events that do occur.

A safe and simple gas mixer for mixing a hydrocarbon containing gas with a gaseous oxidant is disclosed herein. The two gases are introduced directly, and separately, into the bottom of a closed mixing vessel where they form bubbles that are mixed as they rise to the top of the vessel. Mixing results in a homogeneous swarm of bubbles that exit the liquid as non-flammable compositions of hydrocarbon and oxygen, useful for applications involving catalytic oxidation of hydrocarbons, including the oxidative dehydrogenation (ODH) of ethane into ethylene.

Provided is a gas mixer for mixing a hydrocarbon containing gas and a gaseous oxidant comprising a closed mixing vessel flooded with a non-flammable liquid and having a top end, a bottom end, internal mixing means, injection and removal points for non-flammable liquid, inputs for each of the hydrocarbon containing gas and the gaseous oxidant at or near the bottom of the closed mixing vessel, and an outlet at the top of the closed mixing vessel for the removal of a substantially homogeneous mixture of hydrocarbon and oxygen in a ratio that corresponds to the relative amounts of hydrocarbon containing gas and gaseous oxidant introduced into the bottom of the closed mixing vessel.

Provided is a method for mixing a hydrocarbon containing gas with a gaseous oxidant comprising introducing a hydrocarbon containing gas and a gaseous oxidant into the bottom of a closed mixing vessel flooded with a non-flammable liquid and in a ratio that once combined falls outside of the flammability envelope, and recovering a homogeneous mixture of the previously introduced hydrocarbon containing gas and gaseous oxidant.

Provided is a process for the oxidative dehydrogenation of ethane to ethylene comprising the safe mixing of an ethane containing gas with a gaseous oxidant in a closed mixing vessel flooded with a non-flammable liquid, passing the mixture of the ethane containing gas and the gaseous oxidant through a heat exchanger to raise the temperature to at least 250° C., introducing the heated mixture into an ODH reactor containing an ODH catalyst, removing ethylene, unconverted ethane and the various byproducts that include CO, CO₂, H₂O, CH₃COOH, minimal hydrocarbons, and possibly O₂ from the ODH reactor and directing them to a quench tower to remove CH₃COOH and H₂O, the remainder subjected to passage through at least one amine wash to remove CO₂, and finally removal of methane after passage through a demethanizer. The remaining mixture, containing unreacted ethane, ethylene and other hydrocarbons, may be recycled back to go through the process again, starting with mixing with a gaseous oxidant in the closed mixing vessel.

### BRIEF DESCRIPTION OF THE DRAWINGS

FIG. 1-Schematic of the gas mixer disclosed herein.

FIG. 2A-Cross-sectional side view of a section of the gas mixer—Indicated by the dashed box in FIG. 1—in an embodiment where the internal mixing means comprise random packing.

FIG. 2B-Cross-sectional side view of a section of the gas mixer-indicated by the dashed box in FIG. 1—in an embodiment where the internal mixing means comprise structured packing.

FIG. 2C-.Cross-sectional side view of the section of the gas mixer—indicated by the dashed box in FIG. 1—in an embodiment where the internal mixing means comprise an impeller.

FIG. 3-Schematic representation of the gas mixer in an embodiment where the supply nozzles enter at the top end of the gas mixer.

FIG. 4A-Cross sectional view of a portion of the gas mixer in an embodiment where the supply nozzles comprise circular concentric spargers.

FIG. 4B-Cross-sectional top view taken through dashed line from FIG. 4A of the gas mixer, in an embodiment where the supply nozzles comprise circular concentric spargers.

### GAS MIXER

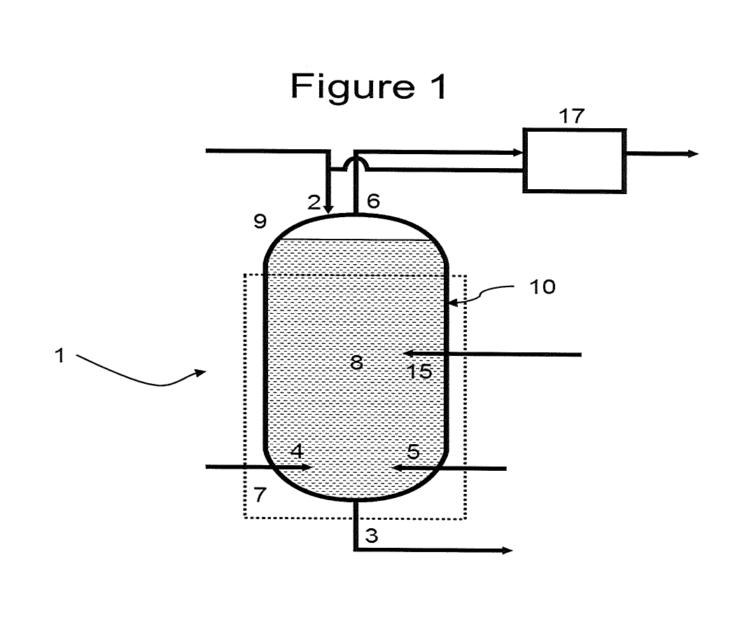

A schematic representation of an embodiment of the gas mixer of the present disclosure is shown in FIG. 1. The gas mixer 1 comprises a closed mixing vessel 10 having a top end 9 and a bottom end 7. The closed mixing vessel 10 is flooded with a non-flammable liquid, the choice of which depends on the application for which the mixed gas is to be used. Non-flammable liquid may be added to the closed mixing vessel 10 via a nozzle or inlet 2 located at the top end 9, while non-flammable liquid may be removed from the outlet 3 located at the bottom end 7. Construction of the mixing vessel 10 can be accomplished with a variety of materials including stainless steel, carbon steel, and any other material chemically compatible with the hydrocarbon to be mixed. Furthermore, the lining of mixing vessel 10 may be coated with a spark suppressing material such as Teflon, sapphire, or oxide-based ceramic liners or the like.

Hydrocarbon containing gas may be introduced into the closed mixing vessel 10 through the hydrocarbon containing gas supply nozzle 4, while the gaseous oxidant may be introduced via gaseous oxidant supply nozzle 5. The hydrocarbon containing gas supply nozzle 4 and the gaseous oxidant supply nozzle 5 cooperate with the closed mixing vessel 10 in a way so that introduction of the gases directly into the non-flammable liquid occurs at or near the bottom end 7 of the closed mixing vessel 10. For the purposes of this disclosure, the term “nozzle” refers simply to the point where contact between the gases and the non-flammable liquid within the closed mixing vessel 10 first occurs, and can include any means known within the art. While not essential, the hydrocarbon containing gas supply nozzle 4 and the gaseous oxidant supply nozzle 5 are ideally orientated such that streams of the hydrocarbon containing gas and the gaseous oxidant impinge upon one another immediately upon entering the mixer. The introduced gases rise and are mixed through mixing zone 8 and are available for removal after exiting the non-flammable liquid at the top of the closed mixing vessel 10 through the mixed gas removal line 6. As the term suggests, non-flammable liquids used to flood the closed mixing vessel 10 must not be flammable. That is, the non-flammable liquid must not be capable of igniting or burning. Suitable non-flammable liquids include water, ethylene glycol, silicon oils, and carbon tetrachloride. One embodiment comprises water as the non-flammable liquid. While any non-flammable liquid may be used with the various embodiments disclosed herein, it is important to consider that mixed gas removed from the gas mixer 1 will comprise the hydrocarbon containing gas, gaseous oxidant, and in some instances carry over of non-flammable liquid. For this reason, selection of a non-flammable liquid must consider any potential effects the carry over may have on downstream applications. Catalysts used for oxidative reactions may be sensitive to catalytic poisoning by specific non-flammable liquids that are carried over in a gaseous state.

The temperature, along with the pressure, play a role in determining what fraction of the non-flammable liquid may enter the gaseous state, joining the hydrocarbon and oxygen gas present in bubbles that are mixing and rising to the top end of the closed mixing vessel 10. The temperature and pressure can be controlled to minimize the carryover of non-flammable liquid into the gas mixture leaving through mixed gas removal line 6. Temperature control using a heater, within or without the closed mixing vessel 10, is contemplated for use with the present disclosure. Heaters for use in mixing vessels similar to that of the present disclosure are well known. In one embodiment the closed mixing vessel 10 is temperature controlled using a heater that is external to the closed mixing vessel 10. In another embodiment the closed mixing vessel 10 is temperature controlled using a heater that is located within the closed mixing vessel 10.

In some instances it may be desirable, for recycling purposes, to include a secondary hydrocarbon containing gas supply nozzle or product supply nozzle 15. For example, some oxidative reactions are not as efficient as others and may include conversion rates below an acceptable level. In those cases, it may be desirable to send a product line containing product and unreacted hydrocarbon back to start the oxidative reaction process again, with the intent of maximizing conversion of the starting hydrocarbon—the hydrocarbon originally mixed in the gas mixer before passage through an oxidative process. The product stream, similar to and containing unreacted starting hydrocarbon, would need to be mixed with oxidant before entering the reactor. If the product contained in the product stream is more reactive to oxygen than the starting hydrocarbon, it would be safer to introduce the product stream into the reactor at a point where the oxygen is already partially mixed and diluted. To this end the secondary hydrocarbon containing gas supply nozzle 15 should be at a position distant from the gaseous oxidant supply nozzle 5. The position of the secondary hydrocarbon containing gas supply nozzle 15 is not critical, provided it is in a position where the oxygen present in the closed mixing vessel 10 has begun mixing with the hydrocarbon containing gas, and there is sufficient residence time for the product gas to mix thoroughly with the added oxygen and hydrocarbon containing gases. Ideally, the position of the secondary hydrocarbon containing gas supply nozzle is near a point equidistant from the gaseous oxidant supply nozzle 5 and the point where mixed gas removal line 6 leaves the top end 9 of the closed mixing vessel 10. The secondary hydrocarbon containing gas supply nozzle 15 may also be used as an additional input location for the introduction of the hydrocarbon containing gas. In one embodiment, there is a secondary hydrocarbon containing gas supply nozzle 15 for introducing a product stream from an oxidative process or additional hydrocarbon containing gas into the closed mixing vessel 10 at a point distant from gaseous oxidant supply nozzle 5.

In instances where there is recycling of an oxidative process such that a product line is fed back to the gas mixer 1 for introduction into the closed mixing vessel 10 via the secondary hydrocarbon containing gas supply nozzle 15, it is contemplated that heat from the product line may be used in temperature control of the closed mixing vessel 10. The heat provided from an oxidative process, for example ODH, may be used in this fashion and would therefore assist in reducing the cost associated with providing heat through an internal or external heater. In another embodiment, the closed mixing vessel 10 is temperature controlled using heat from a product line leaving an exothermic oxidation process.

### Internal Mixing Means

The efficiency of mixing of the gases within zone 8 is dependent upon, among other things, the residence time and the frequency of interactions between bubbles of gas. In other words, how often do bubbles collide, break and reform together, permitting mixing of the gas compositions from each of the bubbles which combine to form a homogeneous mixture. While mixing can occur naturally given sufficient time, it is not likely that a homogeneous mixture will be produced without internal mixing where collisions between bubbles are promoted. Without internal mixing the vessel would need to be of such height as to be not economically feasible. Means for promoting mixing are well known in the art and include use of a static mixers, random packing, structured packing, and impellers.

Static mixers promote mixing by creating a multitude of tortuous pathways that increase the distance that bubbles need to travel to reach the top of the vessel and consequently static mixers act partly by increasing the residence time. Also, the pathways comprise limited space that results in an increased probability that bubbles collide and ultimately mix to combine their gaseous contents. In an embodiment, the internal mixing means comprises a static mixer.

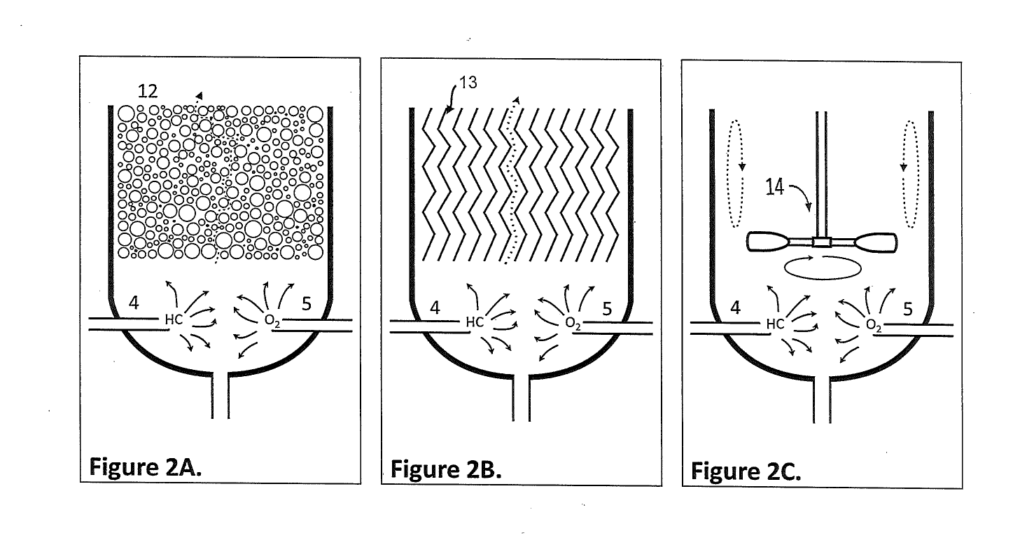

Random and structured packing act similar to static mixers in that they provide for increased residence time and probability of interaction between bubbles by creation of a plethora of winding pathways. Random packing involves filling at least a part of the closed mixing vessel 10 with a packing material that comprises objects 12 of varying shape and size (FIG. 2A) that create random pathways for the bubbles to follow as they rise to the top (see dashed arrow in FIG. 2A). An example of commonly used random packing is glass beads of varying diameter. In another embodiment, the internal mixing means comprised a packed bed.

Structured packing also increases residence time and probability of contact between bubbles, but differs from random packing in that the structured packing has an ordered arrangement so that most of the pathways are of a similar shape and size (see dashed arrows in FIG. 2B). For example, use of corrugated metal plates 13 (FIG. 2B) provides a structured, as opposed to random, array of pathways. In another embodiment, the internal mixing means comprises a structured bed. FIGS. 2A and 2B are provided as simplified examples for random and structured packing and should not be seen to limit the present disclosure in any way. Also, while not shown in the Figures, random and structured packing are supported within the gas mixer using means known in the art.

The present disclosure also contemplates the use of power driven mixers, which can promote interactions by creating flow within the vessel. Impellers include a rotating component 14 (direction of rotation shown by solid circular arrow), driven by a motor, that may force the non-flammable liquid, and associated bubbles of gas, to the outside wall and away from the center of rotation. Impellers can create axial flow or radial flow depending upon design, and can be further sub-typed as propellers, paddles, or turbines (see dotted arrows on either side of the gas mixer in FIG. 2C). Furthermore, the position of the impeller may be subject to change through vertical movement throughout the mixing zone 8. Motor driven pumping of an impeller further improves mixing. In another embodiment the closed mixing vessel 10 further comprises an impeller.

Similar to the closed mixing vessel 10, the internal mixing means, whether a static mixer, random or structured packing, or an impeller may be comprised of any material that is chemically compatible with the hydrocarbon to be mixed.

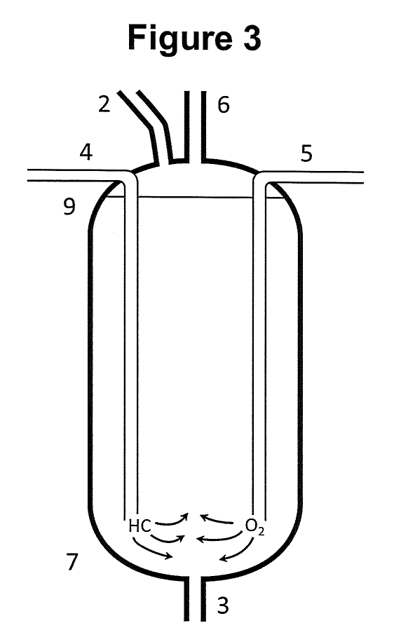

The shape and design of the closed mixing vessel 10 impacts the residence time. The overall shape of the vessel is not critical, but the distance between where the gas enters and exits the mixing zone 8 is. The point of first contact between the gases and the water in the closed mixing vessel 10 should be a distance from the top that allows for a residence time that permits complete mixing before removal. In another embodiment, the ideal entry point is near the bottom of the vessel. Where the lines containing the gas enter the vessel is not important, provided the nozzle—the point where the gas contacts the water in the vessel—is in the position where residence time is sufficient. For example, in FIG. 3 the lines feeding the hydrocarbon containing gas and the gaseous oxidant enter the closed mixing vessel 10 near the top end 9 but the nozzles 4 and 5 introduce the gases near the bottom end 7.

Another consideration for the optimum mixing of the gases is the surface area over which the gases are dispersed. A larger surface area of dispersion promotes better mixing. While injection through a single inlet is feasible, provided sufficient residence time, more thorough mixing occurs when a larger number of smaller bubbles are dispersed over a larger surface area. Having multiple hydrocarbon containing gas supply nozzles and multiple gaseous oxidant supply nozzles allows each of the gases to be introduced in multiple locations. Conversely, a single nozzle may comprise multiple exit points where gas can enter the vessel, effectively dispersing the gas over a greater surface area compared to dispersion from a nozzle with a single exit point. In an embodiment, at least one of the hydrocarbon containing gas supply nozzle 4 and the gaseous oxidant supply nozzle 5 comprise a sparger.

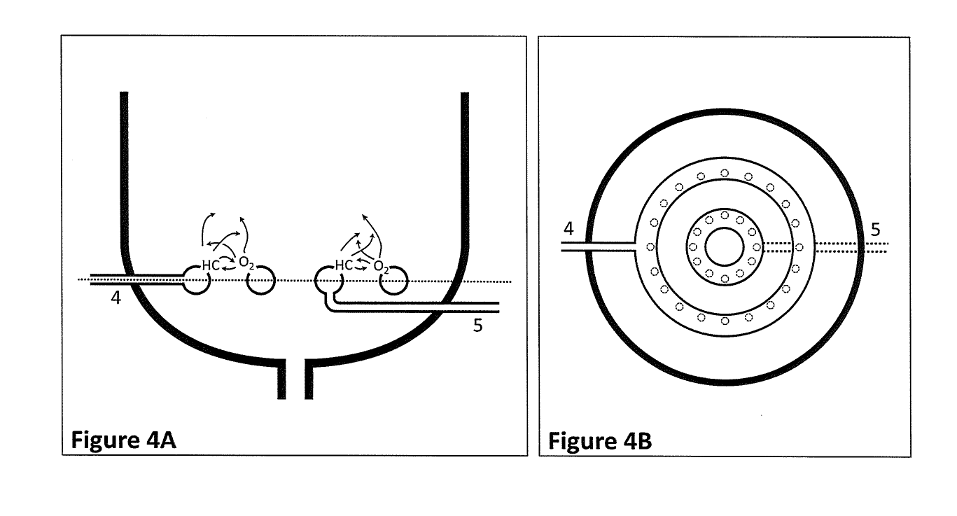

In another embodiment, the hydrocarbon containing gas supply nozzle 4 and the gaseous oxidant supply nozzle 5 are arranged as spargers in the form of concentric rings. For example, in FIGS. 4A and 4B the hydrocarbon containing gas supply nozzle 4 comprises a circular sparger that is larger than and surrounds the gaseous oxidant supply nozzle 5, which is also circular and fits inside the hydrocarbon containing gas supply nozzle 4. Furthermore, the exit points for the hydrocarbon containing gas and the gaseous oxidant from their respective nozzles are arranged such that the streams of gas impinge on one another, initiating mixing as early as possible after introduction into the mixer (see FIG. 4A). The arrangement of the gas supply nozzles is not limited to examples provided here. A series of concentric rings, with alternating oxygen and hydrocarbon containing gas supply nozzles, is also contemplated.

### Emergency Shutdown

Another embodiment relates to emergency shutdown procedures common to oxidative reaction processes. It is well known that when undesirable conditions occur in an oxidative reaction process an emergency shutdown procedure can be initiated to limit damage to equipment, reduce likelihood of personal injury, and prevent or minimize release of chemicals into the surrounding environment. Known emergency shutdown procedures include the cessation of adding reactants while at the same time providing a flow of an inert material, such as nitrogen, to the reaction zone to displace the reactants from the reactor. See U.S. Pat. No. 7,414,149 to DeCourcy and Le, assigned to Rohm and Haas Company, for an example.

In some embodiments, it is contemplated that for an additional safety component an inert material inlet, located near the top end and above the liquid level, may be included for the introduction of a flow of an inert material. In addition, a suppression outlet leading to any known explosion suppression system may be included near the top end of the gas mixer. When an unsafe operating condition is detected at any point in the oxidative process, flow of an inert material through the inert material inlet can be initiated while the suppression outlet can be opened. These events can be coordinated with a reduction or termination of the hydrocarbon and oxidant reactants. The end result is that any mixed gases within the mixer are displaced to the explosion suppression system or to downstream components of the oxidative process. The flow of inert material acts as diluent and promotes movement in a single direction so that backflow of materials from the oxidation reactor into the gas mixer are prevented.

In an embodiment, the gas mixer further comprises an inert material inlet, located near the top end of the gas mixer, for introducing an inert material into the gas mixer above the level of the non-flammable liquid, and a suppression outlet for removing gaseous mixtures, located near the top end of the gas mixer and leading to an explosion suppression system.

### Method for Mixing a Hydrocarbon Containing Gas and a Gaseous Oxidant

The present disclosure is relevant for applications that require the mixing of a hydrocarbon containing gas with a gaseous oxidant. It is well known that gaseous compositions containing a hydrocarbon and oxygen in ratios that fall within the flammability envelope are potentially hazardous. An ignition event, such as a spark, can ignite the mixture and potentially lead to an explosion. While applications that require mixing of hydrocarbons and oxygen normally do so with ratios that are safe and not susceptible to ignition there are moments during initial mixing where heterogeneous pockets of unfavorable hydrogen/oxygen compositions exist and may ignite if a spark occurs.

The present disclosure seeks to provide a method for mixing a hydrocarbon containing gas with a gaseous oxidant that is simple, and safe in that ignition events are unlikely to occur. The method comprises introducing, separately and simultaneously, a hydrocarbon containing gas and a gaseous oxidant directly into a closed mixing vessel having a top end and a bottom end and flooded with a non-flammable liquid, in close proximity to the bottom end, allowing the bubbles of gas to mix while surrounded by the non-flammable liquid, and removing from the top of the vessel, after mixing is complete, a homogeneous mixture of the hydrocarbon containing gas and the gaseous oxidant in a ratio that is outside of the flammability envelope.

In some embodiments, the amount of the gases introduced into the bottom end of the closed mixing vessel 10 will result in a final composition that comprises a ratio of hydrocarbon containing gas to gaseous oxidant that is outside of the flammability envelope. The chosen ratio will depend on the nature of the gases and the application for which the mixture will be used. For example, for an ODH application, the ratio of ethane to oxygen chosen will depend on whether under the proposed ODH reaction conditions the ratio is above the higher explosive limit or below the lower explosive limit. In comparison, the ratio of ethylene to oxygen added to the reactor would be different because ethylene is more reactive than ethane. The temperature of the ODH process to be employed must also be taken into consideration as higher temperatures correspond to a much smaller window of safe ratios of ethane to oxygen. For example, a molar ratio of about 80:20 ethane to oxygen for catalytic ODH would fall above the upper explosive limit, while a ratio of about 1.5:98.5 ethane to oxygen would fall below the lower explosive limit, with each ratio safe enough in that ignition events would not lead to an explosion or flame propagation under ODH reaction conditions. Ratios falling between that—50:50 for example—would be unsafe and potentially flammable/explosive.

The next consideration after determining the desired final ratio of hydrocarbon to oxygen is determining the flow rate at which each gas is added to the bottom of the closed mixing vessel 10. The flow rate of the gases and the corresponding pressure would need to be higher than the pressure of the non-flammable liquid in the closed mixing vessel 10. In the absence of a pressure differential, the gases cannot enter the closed mixing vessel 10 and consequently the mixing zone 8. Furthermore, if the pressure of the non-flammable liquid is higher than the line containing the gas to be introduced there may be, in the absence of a one way valve, flow back of non-flammable liquid into the gas supply lines. This should be avoided.

When determining flow rates, the skilled worker must correlate the flow rates with the pressure and temperature used within the closed mixing vessel 10. The conditions within the closed mixing vessel 10 are chosen to reflect the amount of carryover of non-flammable liquid into the gas mixture removed through mixed gas removal outlet 6. The flow rate of the incoming gases must be sufficient to allow entry into the non-flammable liquid at the predetermined temperature and pressure.

As a further safety precaution, the present disclosure also contemplates the dilution of the gaseous oxidant with non-flammable liquid prior to entry into the closed mixing vessel 10. The prior dilution of the gaseous oxidant permits the saturation of incoming oxygen molecules with molecules of the non-flammable liquid that discourage ignition events igniting any hydrocarbons that interact with the oxygen during the early stages of mixing. Dilution of the gaseous oxidant with non-flammable liquid can be accomplished by directing a non-flammable liquid line into the gaseous oxidant line prior to the gaseous oxidant nozzle. Non-flammable liquid present within the closed mixing vessel 10 that is ejected via outlet 3 may be suitable for this purpose, provided this non-flammable liquid passes through a filter to remove particulate matter prior to introduction into the gaseous oxidant line. In one embodiment, the gaseous oxidant is diluted with non-flammable liquid prior to introduction into the closed mixing vessel 10.

The choice of gas mixer and associated design of the closed mixing vessel should consider the factors discussed above. The gas mixer must allow for a residence time that allows complete, or near complete, mixing to create a homogeneous composition of gas where there are no potentially unsafe pockets of gas with undesirable ratios of hydrocarbon to oxygen.

The final consideration is the removal of the mixed gas from the top of the closed mixing vessel, which can be accomplished with any variety of means for removal well known in the art.

### Oxidative Dehydrogenation

Oxidative dehydrogenation of paraffins to olefins is an alternative to the costly, energy intensive and environmentally unfriendly thermal cracking method currently used. In ODH, a stream of one or more alkanes are passed over a catalyst in the presence of oxygen, to produce corresponding olefins and a variety of byproducts that can be removed in downstream processing steps. Since in ODH the conversion of paraffins to olefins is assisted by a catalyst the required operating temperatures are significantly lower than the temperature required for thermal cracking. Also, for conversion of ethane to ethylene, ODH provides for higher conversion and selectivity rates. Despite these advantages ODH is not employed commercially due to the risk of thermal runaway of the reaction and consequential explosion. This risk is due to the requirement for mixing a hydrocarbon containing gas with oxygen or a gaseous oxidant.

Provided herein is a process for the oxidative dehydrogenation of a paraffin to a corresponding olefin. More specifically, provided herein is a process for oxidative dehydrogenation of ethane into ethylene comprising mixing of ethane and oxygen in a ratio that falls outside of the flammability envelope in a closed mixing vessel, passing the mixture of ethane and oxygen through a heat exchanger to raise the temperature to at least 250° C., introducing the heated mixture into an ODH reactor containing an ODH catalyst to produce ethylene, carbon monoxide, carbon dioxide, water, acetic acid, minimal hydrocarbons, and possibly O₂, directing said products through a quench tower to remove water and acetic acid, directing the residual products through an amine wash to remove carbon dioxide, and finally through a demethanizer to remove methane.

By using the gas mixer and method of mixing a hydrocarbon containing gas and a gaseous oxidant discussed above the inherent risks of catalytic ODH are minimized. The mix of ethane and oxygen entering the reactor is outside the flammability envelope so that thermal runaway and subsequent explosion is not likely. Furthermore, by premixing the gases a user can ensure consistent conversion due to the homogeneous nature of the ethane, oxygen mix.

### EXAMPLES

In a mixture of ethane and oxygen, at 25° C., the upper explosive limit (UEL), defined by a ratio in mole %, of ethane to oxygen, is approximately 62.18 to 32.81—where the balance is in the form of vaporous water—or 1.90 to 1. A model of a theoretical mixer where temperature and pressure are independently controllable was used to predict that a ratio of ethane to oxygen at the UEL can be maintained over a range of temperature and pressure. At each temperature and pressure there is a fraction that includes water that leaves the liquid phase and enters the vapor phase. By controlling temperature and pressure the carryover of vaporous water can be manipulated. Table 1 shows the fraction of the mixture that is water, ethane and oxygen, at various temperatures and pressure. As the temperature and pressure are increased the fraction of water within the gaseous phase decreases.

Furthermore, at the temperature and pressures listed the UEL is not breached. i.e., the mole % of oxygen does not exceed 62.81.

TABLE 1
Calculated Mole Fraction of Ethane, Oxygen, and Water as a
Function Of The Temperature And Pressure

| | Simulation | | | | | |
|---|---|---|---|---|---|---|
| | 1 | 2 | 3 | 4 | 5 | 6 |
| Temperature | 44.7 | 50.4 | 58.6 | 64.0 | 71.7 | 82.2 |
| (° C.) | | | | | | |
| Pressure (bar) | 1.00 | 1.45 | 2.45 | 3.45 | 5.68 | 11.36 |
| | Mass Flow added to the mixer (kg/hr) | | | | | |
| Ethane (C₂H₆) | 35.40 | 35.40 | 35.40 | 35.40 | 35.40 | 35.40 |
| Oxygen (O₂) | 19.87 | 19.87 | 19.87 | 19.87 | 19.87 | 19.87 |
| Water vapour | 3.42 | 3.15 | 2.76 | 2.51 | 2.15 | 1.71 |
| (H₂O) | | | | | | |
| | Mole Fraction (mole %) | | | | | |
| Ethane (C₂H₆) | 0.592 | 0.597 | 0.603 | 0.608 | 0.614 | 0.622 |
| Oxygen (O₂) | 0.312 | 0.315 | 0.318 | 0.321 | 0.324 | 0.328 |
| Water (H₂O) | 0.095 | 0.089 | 0.078 | 0.072 | 0.062 | 0.050 |

What is claimed is:

1. A gas mixer for mixing a hydrocarbon containing gas with a gaseous oxidant, comprising:
i) a closed mixing vessel having a top end, a bottom end, and flooded with a non-flammable liquid;
ii) a liquid supply nozzle for introducing a non-flammable liquid into the closed mixing vessel in close proximity to the top end;
iii) a drain connection for removing non-flammable liquid from the closed mixing vessel located in close proximity to the lowest point of the bottom end;
iv) at least one hydrocarbon containing gas supply nozzle for introducing hydrocarbon containing gas into the closed mixing vessel near the bottom end;
v) at least one gaseous oxidant supply nozzle for introducing gaseous oxidant into the closed mixing vessel near the bottom end;
vi) at least one means within the closed mixing vessel for internal mixing of introduced hydrocarbon containing gas with introduced gaseous oxidant; and
vii) a mixed gas outlet located in close proximity to the uppermost point of the top end;
wherein the level of non-flammable liquid within the closed mixing vessel is at a height sufficient to allow mixing of the introduced hydrocarbon containing gas and the gaseous oxidant before reaching the top end such that bubbles of gas exiting the non-flammable liquid comprise a mixture of hydrocarbon containing gas and gaseous oxidant that is outside the flammability limit.

2. The gas mixer of claim 1 wherein the non-flammable liquid is water.

3. The gas mixer of claim 1 wherein the means for internal mixing is selected from the group comprising:
i) a static mixer;
ii) a packed bed;
iii) a structured bed; and
iv) an impeller.

4. The gas mixer of claim 1, wherein at least one of the hydrocarbon containing gas supply nozzle and the gaseous oxidant supply nozzle is connected to a sparger.

5. The gas mixer of claim 1, wherein at least one of the hydrocarbon gas containing supply nozzle and the gaseous oxidant supply nozzle comprises an aerator nozzle.

6. The gas mixer of claim 1, further comprising:
i) a condenser down-stream of the mixed gas outlet for condensing gaseous contaminants, including gaseous non-flammable liquid, present in the gaseous mixture;
ii) a recycle line for conducting non-flammable liquid from the condenser back to the closed mixing vessel where the non-flammable liquid is reintroduced in close proximity to the top end.

7. The gas mixer of claim 1, further comprising at least one secondary hydrocarbon supply nozzle for introducing into the closed mixing vessel a hydrocarbon containing gas, a product stream via a product recycle line from an oxidation type reaction process, or both at a location distant from the bottom end.

8. The gas mixer of claim 7 wherein the hydrocarbon containing gas is ethane and the oxidative type reaction process is an ODH reaction process.

9. The gas mixer of claim 1, further comprising means for controlling the temperature of the non-flammable liquid in the closed mixing vessel selected from the group comprising:
i) heat from the product recycle line;
ii) an external heater; and
iii) an internal heater.

10. The gas mixer of claim 1, further comprising:
i) an inert material inlet located near the top end for introducing an inert material into the gas mixer above the level of the non-flammable liquid; and
ii) a suppression outlet located near the top end and leading to an explosion suppression system for removing gaseous mixture from above the level of the non-flammable liquid.

11. A method of mixing a hydrocarbon containing gas and a gaseous oxidant within a closed mixing vessel flooded with a non-flammable liquid and having a bottom end, a top end, and internal mixing means, with the resulting gaseous mixture being homogenous and comprising a ratio of hydrocarbon containing gas to gaseous oxidant that is outside the flammability envelope, the method comprising:
i) introducing a hydrocarbon containing gas into close proximity to the bottom end of the closed mixing vessel;
ii) introducing a gaseous oxidant, simultaneously with the hydrocarbon containing gas, in close proximity to the bottom end of the closed mixing vessel; and
iii) recovering a homogenous gaseous mixture of hydrocarbon containing gas and gaseous oxidant in close proximity to the top end of the closed mixing vessel;
wherein the level of non-flammable liquid together with the internal mixing means provide sufficient residence time and promote interactions between bubbles of hydrocarbon containing gas and gaseous oxidant such that the gas exiting the non-flammable liquid comprises a mixture of hydrocarbon containing gas and gaseous oxidant that is outside the flammability limit.

12. The method of claim 11 wherein the non-flammable liquid is water.

13. The method of claim 11 wherein the hydrocarbon containing gas comprises ethane.

14. The method claim 11 wherein the hydrocarbon containing gas comprises methane.

15. The method of claim 11 wherein the resulting gaseous mixture is passed through a condenser to remove gaseous non-flammable liquid, which can be recycled back to the closed mixing vessel, and other gaseous contaminants which can be discarded.

16. The method of claim 11 wherein a product stream from an ODH process, ethane, or a combination of both is introduced into the closed mixing vessel at a location distant from the bottom end.

17. The method of claim 11 wherein the gaseous oxidant is diluted with water prior to introduction into the closed mixing vessel.

18. A process for the oxidative dehydrogenation of ethane to ethylene, the process comprising:
i) mixing an ethane containing gas and a gaseous oxidant in a closed mixing vessel;
ii) passing the mixture from i) through a heat exchanger to raise the temperature to at least 250° C.;
iii) introducing the mixture from ii) into an ODH reactor containing an ODH catalyst, giving a product stream containing unreacted ethane, CO, C₂H₄, CO₂, H₂O, CH₃COOH, minimal hydrocarbons, and possibly O₂;
iv) directing product stream from iii) through a quench tower to remove H₂O and CH₃COOH;
v) directing product stream from iv) through at least one amine wash to remove CO₂; and
vi) directing product stream from v) through a demethanizer (distillation tower) to separate from the product stream.

19. The process of claim 18 wherein the product stream from any of iii), iv), v), or vi) is recycled back to the closed mixing vessel.
